(19) United States
(12) Patent Application Publication
(10) Pub. No.: US 2023/0036375 A1
(43) Pub. Date: Feb. 2, 2023

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Kanagawa (JP); Yasuyuki HOSONO, Kanagawa (JP); Nobuya KITAMURA, Kanagawa (JP); Kenji NAGAMIYA, Kanagawa (JP); Tomohide HIRAGAMI, Kanagawa (JP)

(21) Appl. No.: 17/811,071

(22) Filed: Jul. 7, 2022

(30) Foreign Application Priority Data
Jul. 29, 2021 (JP) 2021-124689

(51) Int. Cl. G16H 40/63

(52) U.S. Cl. CPC G16H 40/63

(57) ABSTRACT

An information processing apparatus includes at least one processor. Input information, which is information input to the information processing apparatus, and biological information of a user are acquired, and association processing of associating information on a physical condition, a symptom, health, feeling, emotion, or a disease name of the user in the input information with the biological information at a point in time corresponding to a point in time when the input information is input, is performed.

### CROSS-REFERENCE TO RELATED APPLICATIONS

The present application claims priority under 35 U.S.C. § 119 to Japanese Patent Application No., 2021-124689 filed on Jul. 29, 2021. The above application is hereby expressly incorporated by reference, in its entirety, into the present application.

### BACKGROUND

### 1. Technical Field

The disclosed technology relates to an information processing apparatus, an information processing method, and an information processing program.

### 2. Description of the Related Art

The following technologies are known as technologies of collecting information on behavior of a user and processing the information on the behavior of the user into useful information for use by the user. For example, WO2012/124259A discloses an information terminal comprising a behavior specifying unit that collects behavior information of a user, a first detection unit that detects a change of the collected behavior information, and an information providing unit that provides information on behavior based on a frequency of the behavior corresponding to the detected change.

JP2014-241044A discloses a food information support device including an acquisition unit that acquires food information of a first user including at least one menu and at least one personal information of the first user, a storage unit that stores a food information control master table including a food information control rule which is a rule for changing the food information including at least one menu associated with each of a plurality of pieces of personal information, an extraction unit that extracts the food information control rule corresponding to the first user with reference to the food information control master table based on the acquired at least one personal information of the first user, and a generation unit that generates customized food information obtained by customizing the food information of the first user based on the extracted food information control rule.

### SUMMARY

In recent years, a wearable device, such as a smart watch, has made it possible to measure biological information, such as a heartbeat, a blood pressure, and electrocardiography, of a user. For example, in a case in which the user receives a medical examination at a hospital, a doctor can make a quick and accurate diagnosis by presenting the biological information measured by the wearable device to the doctor. On the other hand, in a case in which the user is suffering from a certain illness, a case may also happen in which the biological information maintains a normal value or a fluctuation of the biological information is delayed from a subjective symptom even though the user is aware of any symptom. Such a relationship between the subjective symptom and the biological information is also considered to be useful for diagnosis. That is, it is considered that the utility value of the biological information can be enhanced by associating the biological information with the subjective symptom.

The disclosed technology is made in view of the above points, and is to enhance the utility value of biological information.

The disclosed technology relates to an information processing apparatus comprising at least one processor. The processor acquires input information, which is information input to the information processing apparatus, and biological information of a user, and performs association processing of associating information on a physical condition, a symptom, health, feeling, emotion, or a disease name of the user in the input information with the biological information at a point in time corresponding to a point in time when the input information is input.

The input information may include a text input to the information processing apparatus or a selection item selected by using the information processing apparatus. The input information may be information input on an application installed in the information processing apparatus.

The input information may include a search keyword input to a search engine for searching for information on a network. The input information may include a content transmitted to or sent to a specified or unspecified party. The input information may include information on a product or service purchased or a product or service to be purchased by information communication via a network.

The processor may store the input information and the biological information associated with each other in a storage medium. The processor may acquire the biological information from a measurement device that measures the biological information by communicating with the measurement device.

The processor may acquire the input information and the biological information for each of a plurality of the users, and may perform the association processing for each user.

The processor may acquire external input information which is information input to an external device by a third party who is permitted to intervene in the association processing, and may further perform association processing of associating information on a physical condition, a symptom, health, feeling, emotion, or a disease name of the user in the external input information with the biological information at a point in time corresponding to a point in time when the external input information is input.

The processor may acquire a result of examination performed on the user as the biological information.

The disclosed technology relates to an information processing method executed by at least one processor provided in an information processing apparatus, the method comprising acquiring, by the processor, input information, which is information input to the information processing apparatus, and biological information of a user, and performing, by the processor, association processing of associating information on a physical condition, a symptom, health, feeling, emotion, or a disease name of the user in the input information with the biological information at a point in time corresponding to a point in time when the input information is input.

The disclosed technology relates to an information processing program causing at least one processor provided in an information processing apparatus to execute acquiring input information, which is information input to the information processing apparatus, and biological information of a user, and performing association processing of associating information on a physical condition, a symptom, health, feeling, emotion, or a disease name of the user in the input information with the biological information at a point in time corresponding to a point in time when the input information is input.

According to the disclosed technology, it is possible to enhance the utility value of the biological information.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a biological information management system according to an embodiment of the disclosed technology;

FIG. 2 is a diagram showing an example of a hardware configuration of an information processing apparatus according to the embodiment of the disclosed technology;

FIG. 3 is a functional block diagram showing an example of a functional configuration of the information processing apparatus according to the embodiment of the disclosed technology;

FIG. 4 is a diagram showing an example of input information and biological information associated with each other by association processing according to the embodiment of the disclosed technology;

FIG. 5 is a diagram showing an example of a display form of the input information and the biological information associated with each other by the association processing according to the embodiment of the disclosed technology;

FIG. 6 is a flowchart showing an example of a flow of processing performed by executing an association program according to the embodiment of the disclosed technology;

FIG. 7 is a diagram showing an example of a configuration of a biological information management system according to another embodiment of the disclosed technology;

FIG. 8 is a diagram showing an example of a configuration of a biological information management system according to still another embodiment of the disclosed technology;

FIG. 9 is a diagram showing an example of the input information and the biological information associated with each other by the association processing according to another embodiment of the disclosed technology;

FIG. 10 is a diagram showing an example of a configuration of a biological information management system according to still another embodiment of the disclosed technology; and

FIG. 11 is a diagram showing an example of the input information and the biological information associated with each other by the association processing according to still another embodiment of the disclosed technology.

### DETAILED DESCRIPTION

In the following, one example of the embodiments of the disclosed technology will be described with reference to the drawings. It should be noted that the same or equivalent components and portions in the drawings are represented by the same reference numerals, and the overlapping description will be omitted.

### First Embodiment

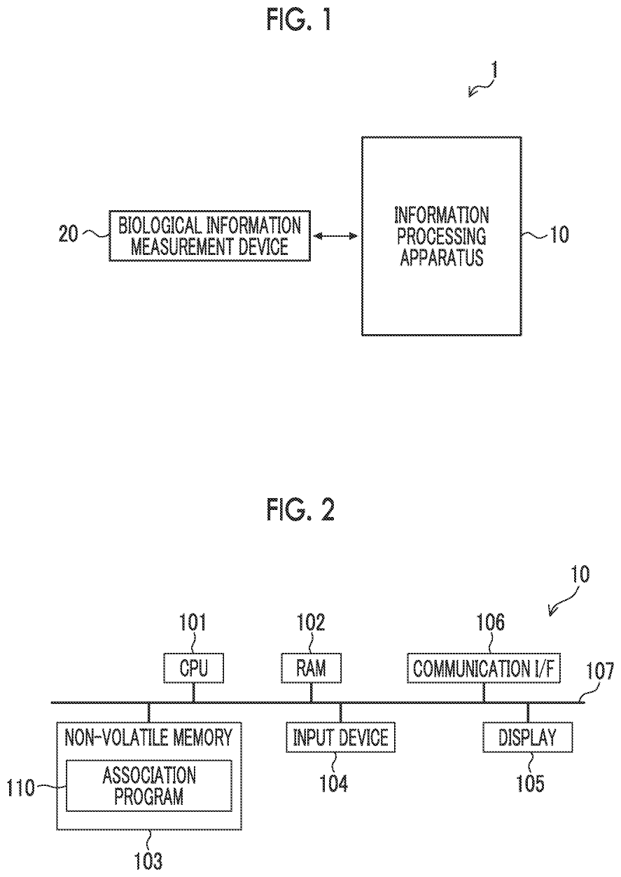

FIG. 1 is a diagram showing an example of a configuration of a biological information management system 1 according to the embodiment of the disclosed technology. A biological information management system 1 includes an information processing apparatus 10 and a biological information measurement device 20.

The information processing apparatus 10 is a communication terminal device used by a person who is a management target of the biological information (hereinafter, referred to as a user). The information processing apparatus 10 may be, for example, a desktop computer, a laptop, a tablet computer, or a smartphone.

The biological information measurement device 20 measures the biological information of the user. The biological information may be, for example, information indicating at least one of a body temperature, a heartbeat, electrocardiography, myoelectricity, a blood pressure, arterial oxygen saturation (SpO2), a blood glucose level, or a lipid level. The biological information measurement device 20 may be a wearable device, such as a smart watch, comprising a sensor that measures these pieces of the biological information. The biological information measurement device 20 has a function of continuously measuring the biological information of the user in accordance with a predetermined time schedule and storing the biological information in an internal memory. The biological information measurement device 20 may measure the biological information for each predetermined period (for example, at intervals of 30 minutes). In addition, the biological information measurement device 20 may always measure the biological information in a case of measuring time-series data, such as an electrocardiographic waveform, as the biological information. The biological information measurement device 20 is communicably connected to the information processing apparatus 10, and transmits the measured biological information to the information processing apparatus 10. It should be noted that the biological information management system 1 may comprise two or more biological information measurement devices that measure different types of the biological information.

FIG. 2 is a diagram showing an example of a hardware configuration of the information processing apparatus 10. The information processing apparatus 10 includes a central processing unit (CPU) 101, a random access memory (RAM) 102, a non-volatile memory 103, a display 105, an input device 104 including a keyboard, a mouse, and the like, and a communication interface 106. The hardware is connected to a bus 107.

The communication interface 106 is an interface for the information processing apparatus 10 to perform communication with the biological information measurement device 20. A communication method may be any of wired or wireless. For wireless communication, for example, a method compliant with an existing wireless communication standard, such as Wi-Fi (registered trademark) and Bluetooth (registered trademark), can be applied. The non-volatile memory 103 is a storage medium, such as a hard disk and a flash memory, and stores an association program 110 described below. The RAM 102 is a work memory for the CPU 101 to execute processing. The CPU 101 loads the association program 110 stored in the non-volatile memory 103 into the RAM 102, and executes the processing in accordance with the association program 110. The CPU 101 is an example of a “processor” according to the disclosed technology. The non-volatile memory 103 is an example of a “storage medium” according to the disclosed technology.

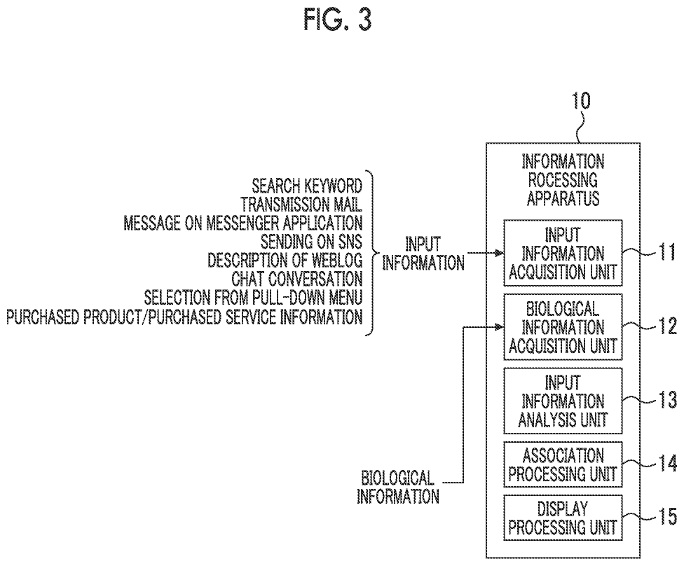

FIG. 3 is a functional block diagram showing an example of a functional configuration of the information processing apparatus 10. The information processing apparatus 10 includes an input information acquisition unit 11, a biological information acquisition unit 12, an input information analysis unit 13, an association processing unit 14, and a display processing unit 15. By the CPU 101 executing the association program 110, the CPU 101 functions as the input information acquisition unit 11, the biological information acquisition unit 12, the input information analysis unit 13, the association processing unit 14, and the display processing unit 15.

The input information acquisition unit 11 acquires input information input to the information processing apparatus 10. The input information can include a text input to the information processing apparatus 10 or a selection item selected by using the information processing apparatus 10. The input information may be information input on various applications installed in the information processing apparatus 10.

The text as the input information may be, for example, a search keyword input to a search engine for searching for information on a network. In addition, the text as the input information may be a text transmitted or sent to a specified or unspecified party, such as a transmission mail, a message on a messenger application, sending on social networking service (SNS), description of a weblog, or a chat conversation. The selection item as the input information may be an item selected by using a graphical user interface (GUI) for selecting from a plurality of options, such as a pull-down menu or a radio button. In addition, the input information may be information (for example, a category of the product or service, a product name, or a service name) on a product or service purchased or a product or service to be purchased from an electronic commerce (EC) site by information communication via the network by using a web browser installed in the information processing apparatus 10. It should be noted that the input information acquisition unit 11 uses the product or service viewed for the purchase of the product or service on the EC site as the product or service to be purchased, and acquires the information on the product or service as the input information. The input information acquisition unit 11 may acquire a combination of any two or more of information described above as the input information.

The input information acquisition unit 11 acquires the input information by linking with various applications, such as the web browser, an e-mail application, a messenger application, and an SNS application installed in the information processing apparatus 10. The input information acquisition unit 11 provides input date and time information indicating the date and time when the input information is input to the information processing apparatus 10 to the input information and stores the input information in the non-volatile memory 103.

The biological information acquisition unit 12 acquires the biological information of the user measured by the biological information measurement device 20. The biological information acquisition unit 12 acquires the biological information from the biological information measurement device 20 by communicating with the biological information measurement device 20. In a case in which the biological information is intermittently measured by the biological information measurement device 20, the biological information acquisition unit 12 may acquire the biological information each time the measurement is performed by the biological information measurement device 20. In a case in which the biological information is time-series data, such as the electrocardiographic waveform, the biological information acquisition unit 12 may acquire the time-series data over a predetermined period (for example, 1 minute) as the biological information in one measurement. In addition, in a case in which the time-series data, such as the electrocardiographic waveform, is always measured by the biological information measurement device 20, the biological information acquisition unit 12 may always acquire the time-series data.

The input information analysis unit 13 analyzes the input information acquired by the input information acquisition unit 11 and specifies the input information satisfying a predetermined condition in the acquired input information. Specifically, the input information analysis unit 13 specifies the input information including a content related to a physical condition, a symptom, health, feeling, emotion, or a disease name of the user in the acquired input information as the input information satisfying the predetermined condition.

The input information analysis unit 13 may specify the input information satisfying the predetermined condition by determining whether or not a word registered in advance as a word related to the physical condition, the symptom, the health, the feeling, the emotion, or the disease name of the user is included in the text or the selection item as the input information. For example, in a case in which the input information is the search keyword input to the search engine, in a case in which the word registered in advance, such as “fever”, “cough”, “headache”, “stomach ache”, “dizzy”, “cold”, “cannot sleep”, “anxiety”, “feel depressed”, “feel listless”, “get annoyed”, “influenza”, “novel coronavirus”, or “COVID-19”, is included in the search keyword, the input information analysis unit 13 may specify the search keyword as the input information satisfying the predetermined condition. In addition, for example, in a case in which the input information is the message input on the messenger application, in a case in which the registered word described above is included in the message, the input information analysis unit 13 may determine that the message satisfies the predetermined condition. The word related to the physical condition, the symptom, the health, the feeling, the emotion, or the disease name of the user may include onomatopoeia, such as “weep”, “disgusted”, or “pit-a-pat”.

In addition, in a case in which the input information is the information on the product purchased or the product to be purchased by the information communication via the network, in a case in which the product is a medical product, such as medicine, the input information analysis unit 13 may specify the information on the product as the input information satisfying the predetermined condition. In addition, in a case in which the input information is the information on the service purchased or the service to be purchased by the information communication via the network, in a case in which the service is related to health service or medical service aimed at maintaining, promoting, or recovering of the health, or eliminating, ameliorating, or alleviating the symptom, the input information analysis unit 13 may specify the input information including the information on the service as the input information satisfying the predetermined condition.

The association processing unit 14 performs the association processing of associating the input information satisfying the predetermined condition specified by the input information analysis unit 13 with the biological information at a point in time corresponding to a point in time when the input information satisfying the predetermined condition in the biological information acquired by the biological information acquisition unit 12 is input. The “biological information at the point in time corresponding to the point in time when the input information is input” is the biological information measured within a predetermined period before and after the point in time when the input information is input, and is, typically, the biological information measured at a point in time closest to the point in time when the input information is input. The association processing unit 14 stores the input information and the biological information associated with each other by the association processing in the non-volatile memory 103.

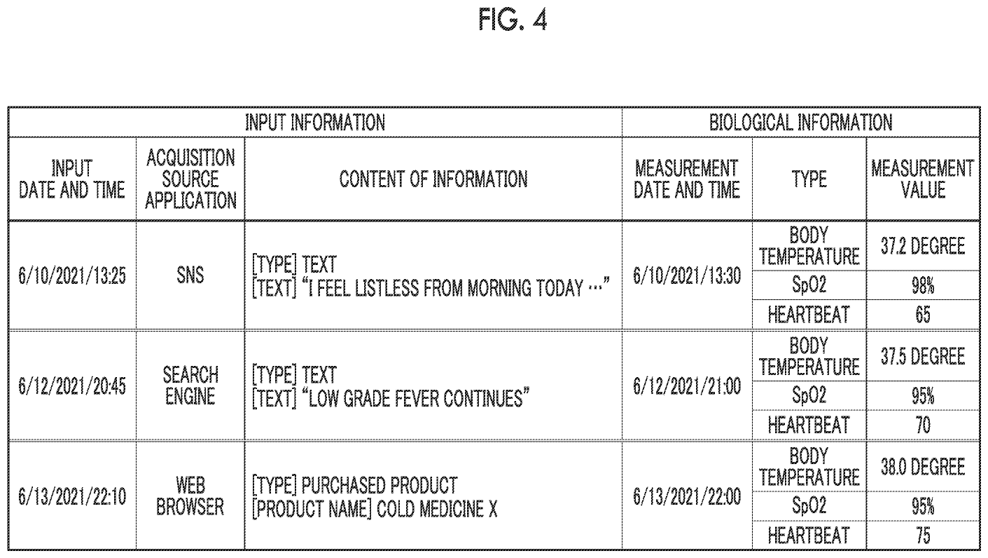

FIG. 4 is a diagram showing an example of the input information and the biological information associated with each other by the association processing. FIG. 4 shows a case in which measurement values of the body temperature, the SpO2, and the heartbeat as the biological information are associated with each input information. As shown in FIG. 4, the input information is provided with the input date and time information indicating the date and time when the input information is input and information indicating a type of the application which is an acquisition source of the input information. In addition, as the content of the input information, a type of information (text/purchased product) and the content of the information (text content/product name) are recorded. In addition, measurement date and time information indicating the date and time when the measurement is performed is provided to each measurement value as the biological information. The measurement date and time corresponds to the input date and time of the input information. FIG. 4 shows the following cases (1) to (3).

(1) The input information analysis unit 13 specifies a text including the word of “feel listless” input on the SNS application installed in the information processing apparatus 10 as the input information satisfying the predetermined condition. The association processing unit 14 associates the text with the measurement values of the body temperature, the SpO2, and the heartbeat as the biological information measured at a point in time (13:30 on Jun. 10, 2021) corresponding to a point in time (13:25 on Jun. 10, 2021) when the text is input (upper part of FIG. 4).

(2) The input information analysis unit 13 specifies a text including the search keyword of “low grade fever continues” input to the search engine as the input information satisfying the predetermined condition. The association processing unit 14 associates the text with the measurement values of the body temperature, the SpO2, and the heartbeat as the biological information measured at a point in time (21:00 on Jun. 12, 2021) corresponding to a point in time (20:45 on Jun. 12, 2021) when the text is input (middle part of FIG. 4).

(3) The input information analysis unit 13 specifies the product name of “cold medicine X” purchased by the information communication via the network using the web browser installed in the information processing apparatus 10 as the input information satisfying the predetermined condition. The association processing unit 14 associates the product name with the measurement values of the body temperature, the SpO2, and the heartbeat as the biological information measured at a point in time (22:00 on Jun. 13, 2021) corresponding to a point in time (22:10 on Jun. 13, 2021) when the product is purchased (lower part of FIG. 4).

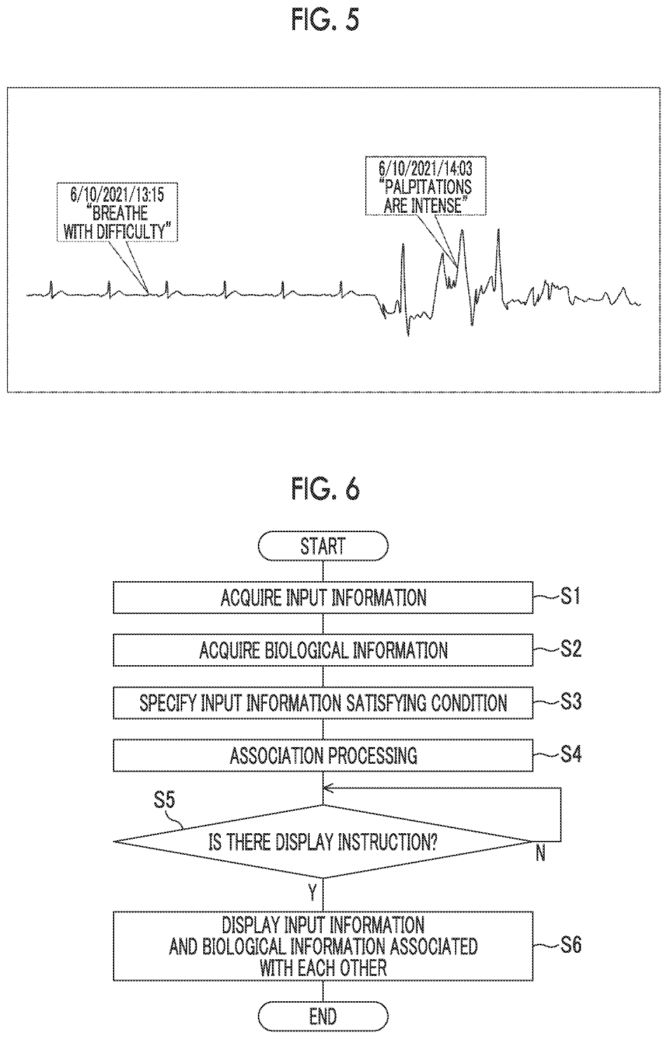

The display processing unit 15 performs processing of displaying the input information and the biological information associated with each other on the display 105 based on a display instruction given by operating the input device 104, for example. The display processing unit 15 may display the input information and the biological information associated with each other in a table format as shown in FIG. 4. In addition, in a case in which the biological information is the time-series data, such as the electrocardiographic waveform, as shown in FIG. 5, the input information and the biological information may be displayed in a format in which the text as the input information is inserted at a corresponding point in time of the time-series data.

FIG. 6 is a flowchart showing an example of a flow of the processing performed by the CPU 101 executing the association program 110. The association program 110 is executed, for example, in a case in which the user gives an instruction to start the processing by operating the input device 104.

In step S1, the CPU 101 functions as the input information acquisition unit 11 to acquire the input information input to the information processing apparatus 10.

In step S2, the CPU 101 functions as the biological information acquisition unit 12 to acquire the biological information of the user measured by the biological information measurement device 20.

In step S3, the CPU 101 functions as the input information analysis unit 13 to specify the input information satisfying the predetermined condition in the input information acquired in step S1. That is, the input information analysis unit 13 specifies the input information including the content related to the physical condition, the symptom, the health, the feeling, the emotion, or the disease name of the user as the input information satisfying the predetermined condition.

In step S4, the CPU 101 functions as the association processing unit 14 to perform the association processing of associating the input information satisfying the predetermined condition specified in step S3 with the biological information at the point in time corresponding to the point in time when the input information satisfying the predetermined condition in the biological information acquired in step S2 is input. The CPU 101 stores the input information and the biological information associated with each other by the association processing in the non-volatile memory 103.

In step S5, the CPU 101 determines whether or not there is the display instruction for the input information and the biological information associated with each other by the association processing in step S4. The display instruction can be given by operating the input device 104. In a case in which it is determined that the display instruction is given, in step S6, the CPU 101 functions as the display processing unit 15 to display the input information and the biological information associated with each other on in the step S4 the display 105.

As described above, the information processing apparatus 10 according to the embodiment of the disclosed technology acquires the input information, which is the information input to the information processing apparatus, and the biological information of the user, and performs the association processing of associating the information on the physical condition, the symptom, the health, the feeling, the emotion, or the disease name of the user in the input information with the biological information at the point in time corresponding to the point in time when the input information is input.

For example, in a case in which the user is aware of an abnormality in the physical condition, it is assumed that the user performs a web search using a word indicating the subjective symptom as the search keyword, complains the subjective symptom to the specified or unspecified party via the messenger application and the SNS, or purchases the medicine in accordance with the subjective symptom. The information processing apparatus 10 according to the present embodiment associates the input information input in accordance with the subjective symptom of the user with the biological information of the user at the point in time corresponding to the point in time when the input information is input. Since it is assumed that the input information includes the content indicating the subjective symptom of the user, it is possible to associate the biological information with the information indicating the subjective symptom. As a result, it is possible to enhance the utility value of the biological information.

For example, in a case in which the biological information acquired by the information processing apparatus 10 is presented to a doctor to receive a diagnosis, an effect of being able to receive a more accurate diagnosis can be expected. In addition, with the information processing apparatus 10 according to the present embodiment, the information that is input on a daily basis on various applications installed in the information processing apparatus 10 is collected as the input information, so that the user can be unconscious about the input of the information on the subjective symptom.

It should be noted that, in the present embodiment, the case has been described in which the input information and the biological information associated with each other are stored in the non-volatile memory 103 provided in the information processing apparatus 10, but the disclosed technology is not limited to this aspect. The input information and the biological information associated with each other may be stored in a storage medium installed outside the information processing apparatus 10. For example, the input information and the biological information may be stored in a cloud storage installed in the network.

In addition, in the present embodiment, the case has been described in which the information processing apparatus 10 acquires all of the biological information measured by the biological information measurement device 20, but the disclosed technology is not limited to this aspect. Only the biological information at the point in time corresponding to the point in time when the input information satisfying the predetermined condition is input may be acquired from the biological information measurement device 20, and the association processing may be performed with respect to the acquired biological information.

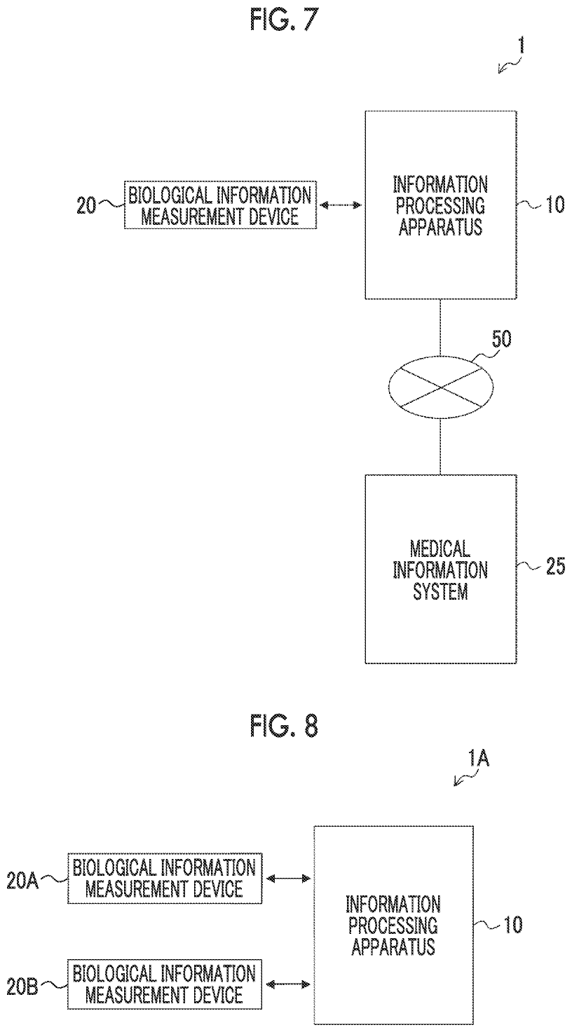

In addition, as shown in FIG. 7, the biological information of the user may be acquired from a medical information system 25 connected to the information processing apparatus 10 via a network 50. The medical information system 25 is a system managed by a medical institution, such as a hospital. The medical information system 25 stores a result of examination performed on the user in the hospital. The biological information acquisition unit 12 acquires the result of examination of the user stored in the medical information system 25 as the biological information of the user.

### Second Embodiment

FIG. 8 is a diagram showing an example of a configuration of a biological information management system 1A according to a second embodiment of the disclosed technology. The biological information management system 1A includes the information processing apparatus 10 and biological information measurement devices 20A and 20B. The biological information measurement devices 20A and 20B measure the biological information of different users. In the following description, a measurement target of the biological information by the biological information measurement device 20A is a user A, and a measurement target of the biological information by the biological information measurement device 20B is a user B. The biological information measured by the biological information measurement devices 20A and 20B is provided with user identification information for identifying the user which is the measurement target of the own device or device identification information for identifying the biological information measurement device.

The information processing apparatus 10 according to the present embodiment acquires the input information and the biological information for each of the user A and the user B, and performs, for each user, the association processing of associating the input information with the biological information. In the following, the details of the information processing apparatus 10 according to the present embodiment will be described.

The input information acquisition unit 11 according to the present embodiment acquires the input information for each of the user A and the user B. That is, the input information acquired by the input information acquisition unit 11 includes information on the user A and information on the user B. It should be noted that the input information may be input to the information processing apparatus 10 by any one of the user A or the user B. For example, in a case in which the user B is a young child or an elderly person and the user A is a guardian or a caregiver of the user B, it is assumed that the input information may be input to the information processing apparatus 10 only by the user A. In this case, for example, the search keyword input by the user A to the search engine related to the user B, the text transmitted or sent by the user A related to the user B, and the product purchased by the user A related to the user B are the input information on the user B.

The biological information acquisition unit 12 according to the present embodiment acquires the biological information of the user A measured by the biological information measurement device 20A and the biological information of the user B measured by the biological information measurement device 20B.

The input information analysis unit 13 according to the present embodiment analyzes the input information acquired by the input information acquisition unit 11 and specifies, for each user, the input information satisfying the predetermined condition in the acquired input information. Specifically, the input information analysis unit 13 specifies the input information including the content related to the physical condition, the symptom, the health, the feeling, the emotion, or the disease name of the user A as the input information on the user A satisfying the predetermined condition. Similarly, the input information analysis unit 13 specifies the input information including the content related to the physical condition, the symptom, the health, the feeling, the emotion, or the disease name of the user B as the input information on the user B satisfying the predetermined condition.

In the present embodiment, for each of the user A and the user B, attribute information of each user including the age, the gender, the height, the weight, the medical history, or the like is stored in the non-volatile memory 103. The input information analysis unit 13 specifies whether the input information is related to the user A or the user B by comparing a word indicating the attribute of the user or a word capable of estimating the attribute of the user included in the text as the input information with the attribute information of each user. For example, in a case in which a rate of match of the word indicating the attribute of the user or the word capable of estimating the attribute of the user included in the text as the input information to the attribute information of user A is larger than a rate of match thereof to the attribute information of the user B, the input information is specified as related to the user A.

The association processing unit 14 according to the present embodiment performs, for each user, the association processing of associating the input information satisfying the predetermined condition with the biological information at the point in time corresponding to the point in time when the input information satisfying the predetermined condition is input. That is, the input information on the user A is associated with the biological information measured for the user A. Similarly, the input information on the user B is associated with the biological information measured for the user B. It should be noted that whether the biological information is related to the user A or the user B can be identified by the identification information provided to the biological information. The association processing unit 14 stores the input information and the biological information associated for each user by the association processing in the non-volatile memory 103.

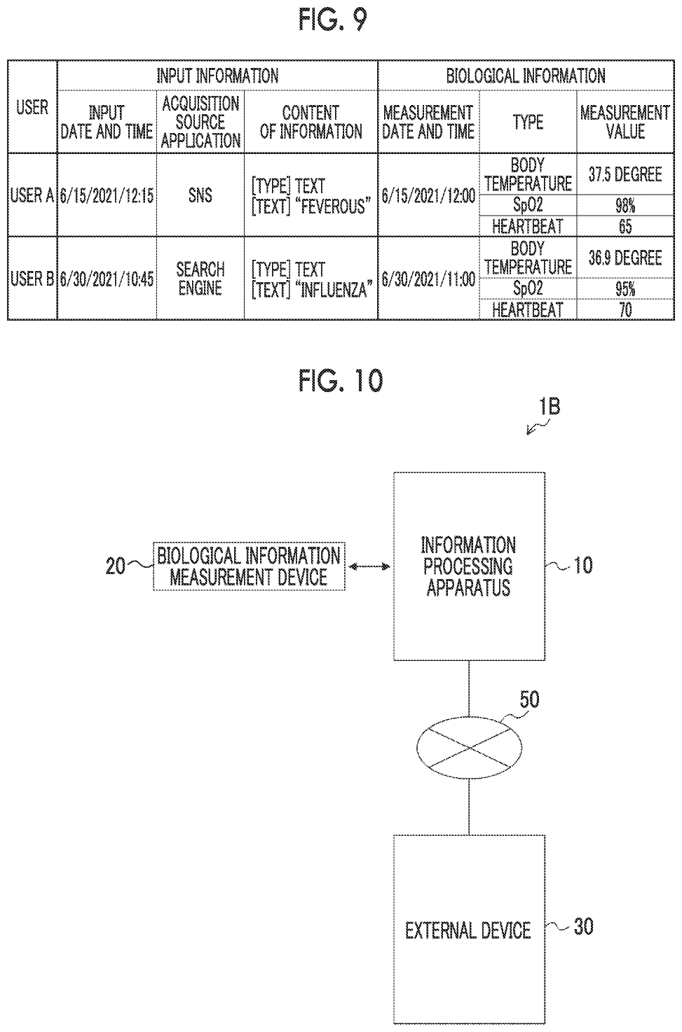

FIG. 9 is a diagram showing an example of the input information and the biological information associated for each user by the association processing. FIG. 9 shows the following cases (4) and (5).

(4) The input information analysis unit 13 specifies the text including a word of “feverous” input on the SNS application as the input information on the user A satisfying the predetermined condition. The association processing unit 14 associates the text with the measurement values of the body temperature, the SpO2, and the heartbeat as the biological information measured for the user A at a point in time (12:00 on Jun. 15, 2021) corresponding to a point in time (12:15 on Jun. 15, 2021) when the text is input (upper part of FIG. 9).

(5) The input information analysis unit 13 specifies the text including the search keyword of “influenza” input to the search engine as the input information on the user B satisfying the predetermined condition. The association processing unit 14 associates the text with the measurement values of the body temperature, the SpO2, and the heartbeat as the biological information measured for the user B at a point in time (11:00 on Jun. 30, 2021) corresponding to a point in time (10:45 on Jun. 30, 2021) when the text is input (lower part of FIG. 9).

The display processing unit 15 according to the present embodiment performs, for each user, the processing of displaying the input information and the biological information associated with each other on the display 105 based on the display instruction given by operating the input device 104, for example. It should be noted that the input information and the biological information of the user A and the input information and the biological information of the user B may be displayed on the display 105 at the same time.

As described above, the information processing apparatus 10 according to the second embodiment of the disclosed technology acquires the input information and the biological information for each of a plurality of users, and performs the association processing of associating the input information with the biological information for each user. With the information processing apparatus 10 according to the present embodiment, it is possible to centrally manage the biological information for the plurality of users.

In addition, for example, in a case in which the user B is the young child or the elderly person and the user A is the guardian or the caregiver of the user B, it is considered difficult for the user B to input the input information. With the information processing apparatus 10 according to the present embodiment, the user A can also perform the association processing for the user B by inputting the input information on the user B.

It should be noted that, in the present embodiment, the case has been described in which the association processing is performed for two users, but it is also possible to perform the association processing for three or more users by assigning the biological information measurement device to each person.

### Third Embodiment

FIG. 10 is a diagram showing an example of a configuration of a biological information management system 1B according to a third embodiment of the disclosed technology. The biological information management system 1B includes the information processing apparatus 10, the biological information measurement device 20, and an external device 30.

As described above, the information processing apparatus 10 is the communication terminal device used by the user who is the management target of the biological information. On the other hand, the external device 30 is a communication terminal device used by a third party who is permitted to intervene in the association processing by the information processing apparatus 10. The external device 30 is communicably connected to the information processing apparatus 10 via the network 50. The external device 30 may be, for example, a desktop computer, a laptop, a tablet computer, or a smartphone.

The input information acquisition unit 11 according to the present embodiment acquires external input information which is the information input to the external device 30, in addition to the input information which is the information input to the information processing apparatus 10. The external input information is the same information as the input information input to the information processing apparatus 10 except that the information is input to the external device 30. That is, the external input information can include a text input to the external device 30 or a selection item selected by using the external device 30. The external input information may be information input on various applications installed in the external device 30.

The text as the external input information may be, for example, the search keyword input to the search engine for searching for the information on the network. In addition, the text as the external input information may be a text transmitted or sent to a specified or unspecified party, such as the transmission mail, the message on the messenger application, sending on the SNS, description of the weblog, or the chat conversation. The selection item as the external input information may be the item selected by using the GUI for selecting from the plurality of options, such as the pull-down menu or the radio button. In addition, the external input information may be the information on the product or service purchased or the like or the product or service to be purchased from the EC site by the information communication via the network using the web browser installed in the external device 30.

The input information acquisition unit 11 acquires the external input information by linking with various applications, such as the web browser, the e-mail application, the messenger application, and the SNS application installed in the external device 30. The input information acquisition unit 11 may authenticate that the third party using the external device 30 is the person who is permitted to intervene in the association processing by the information processing apparatus 10, and then may acquire the external input information from the external device 30. The input information acquisition unit 11 provides the input date and time information indicating the date and time when the input information is input to the information processing apparatus 10 to the input information and stores the input information in the non-volatile memory 103. In addition, the input information acquisition unit 11 provides the input date and time information indicating the date and time when the external input information is input to the external device 30 to the external input information and stores the external input information in the non-volatile memory 103.

The biological information acquisition unit 12 according to the present embodiment acquires the biological information of the user measured by the biological information measurement device 20.

The input information analysis unit 13 according to the present embodiment analyzes the input information and the external input information acquired by the input information acquisition unit 11 and specifies the information satisfying the predetermined condition in the acquired input information and the external input information. Specifically, the input information analysis unit 13 specifies the information including the content related to the physical condition, the symptom, the health, the feeling, the emotion, or the disease name of the user in the input information and the external input information as the information satisfying the predetermined condition.

Here, it is assumed that the external input information input to the external device 30 includes the information on the third party who uses the external device 30, in addition to the information on the user who is the management target of the biological information. In the present embodiment, for each of the user and the third party, the attribute information of each person including the age, the gender, the height, the weight, the medical history, or the like is stored in the non-volatile memory 103. The input information analysis unit 13 specifies whether the external input information is related to the user or the third party by comparing a word indicating the attribute of the user or the third party or a word capable of estimating the attribute of the user or the third party included in the text as the external input information with the attribute information of each person. For example, in a case in which a rate of match of the word indicating the attribute of the user or the third party or the word capable of estimating the attribute of the user or the third party included in the text as the external input information to the attribute information of user is larger than a rate of match thereof to the attribute information of the third party, the external input information is specified as related to the user.

The association processing unit 14 according to the present embodiment performs the association processing of associating the input information satisfying the predetermined condition specified by the input information analysis unit 13 with the biological information at the point in time corresponding to the point in time when the input information satisfying the predetermined condition in the biological information acquired by the biological information acquisition unit 12 is input. In addition, the association processing unit 14 according to the present embodiment further performs the association processing of associating the external input information satisfying the predetermined condition specified by the input information analysis unit 13 with the biological information at the point in time corresponding to the point in time when the external input information satisfying the predetermined condition in the biological information acquired by the biological information acquisition unit 12 is input. The association processing unit 14 stores pieces of information associated by the association processing in the non-volatile memory 103.

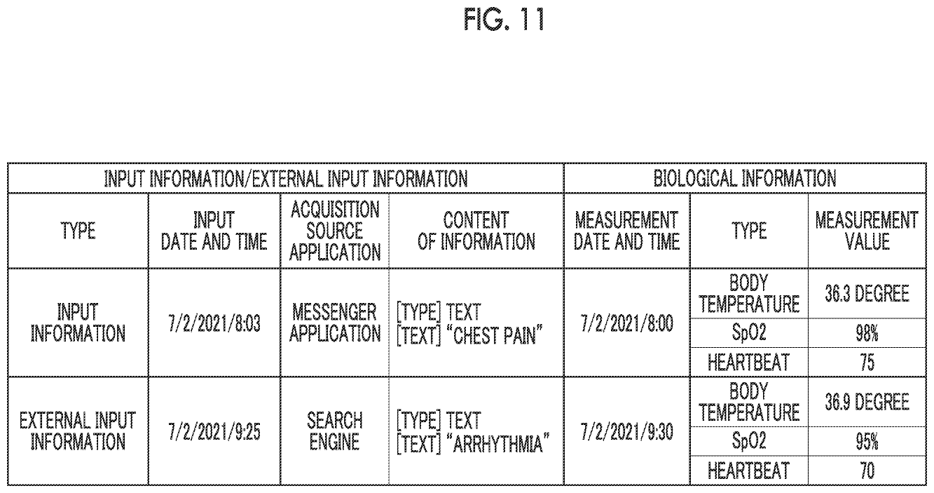

FIG. 11 is a diagram showing an example of the input information/external input information, and the biological information associated with each other by the association processing. FIG. 11 shows the following cases (6) and (7).

(6) The input information analysis unit 13 specifies the text including a word of “chest pain” input on the messenger application installed in the information processing apparatus 10 as the input information satisfying the predetermined condition. The association processing unit 14 associates the text with the measurement values of the body temperature, the SpO2, and the heartbeat as the biological information measured at a point in time (8:00 on Jul. 2, 2021) corresponding to a point in time (8:3 on Jul. 2, 2021) when the text is input (upper part of FIG. 11).

(7) The input information analysis unit 13 specifies the text including a word of “arrhythmia” input to the search engine functioning on the web browser installed in the external device 30 as the external input information satisfying the predetermined condition. The association processing unit 14 associates the text with the measurement values of the body temperature, the SpO2, and the heartbeat as the biological information measured at a point in time (9:30 on Jul. 2, 2021) corresponding to a point in time (9:25 on Jul. 2, 2021) when the text is input (lower part of FIG. 11).

As described above, the information processing apparatus 10 according to the third embodiment of the disclosed technology acquires the external input information input to the external device 30 by the third party who is permitted to intervene in the association processing, and further performs the association processing of associating the information on the physical condition, the symptom, the health, the feeling, the emotion, or the disease name of the user, who is the management target of the biological information, in the acquired external input information with the biological information at the point in time corresponding to the point in time when the external input information is input.

With the information processing apparatus 10 according to the present embodiment, in addition to the input information input by the user himself/herself, the external input information input by the third party other than the user is the target of association with the biological information of the user. For example, in a case in which the user who is the management target of the biological information is a young child or the elderly person, it may be difficult for the user to input the input information by himself/herself. With the information processing apparatus 10 according to the present embodiment, even in such a case, the guardian or the caregiver who is the third party can input the information indicating the symptom of the user to the external device 30, so that the information can be associated with the biological information of the user. With the information processing apparatus 10 according to the present embodiment, for example, the third party located at a location remote from a location of the user can intervene in the association processing of the user, so that the convenience of the user can be enhanced. It should be noted that, as in the biological information management system 1A according to the second embodiment, the technology according to the present embodiment may be applied to the case in which the input information and the biological information are acquired for each of the plurality of users and the association processing is performed for each user, and the third party may be permitted to intervene in the association processing for at least some of the plurality of users.

In each of the embodiments described above, for example, as a hardware structure of processing units that execute various pieces of processing, such as the input information acquisition unit 11, the biological information acquisition unit 12, the input information analysis unit 13, the association processing unit 14, and the display processing unit 15, the following various processors can be used. The various processors include, for example, a programmable logic device (PLD), such as an FPGA, which is a processor of which a circuit configuration can be changed after manufacture, and a dedicated electrical circuit, such as an application specific integrated circuit (ASIC), which is a processor having a dedicated circuit configuration designed to execute specific processing, in addition to the CPU and the GPU which are general-purpose processors that execute software (program) to function as various processing units as described above.

One processing unit may be composed of one of the various processors or may be composed of a combination of two or more processors of the same type or different types (for example, a combination of a plurality of FPGAs or a combination of a CPU and an FPGA). In addition, a plurality of processing units may be composed of one processor.

A first example of the configuration in which the plurality of processing units are composed of one processor is a form in which one processor is composed of a combination of one or more CPUs and software and the processor functions as the plurality of processing units, as represented by the computer, such as a client and a server. A second example thereof is a form in which a processor that realizes all of the functions of a system including the plurality of processing units with one integrated circuit (IC) chip is used, as represented by a system-on-chip (SoC). In this way, various processing units are composed of one or more of the various processors as the hardware structure.

Further, more specifically, an electric circuit (circuitry) obtained by combining circuit elements, such as semiconductor elements, can be used as the hardware structure of the various processors.

In addition, in the embodiments described above, the aspect has been described in which the association program 110 is stored (installed) in advance in the non-volatile memory 103, but the disclosed technology is not limited to this. The association program 110 may be provided in a form of being recorded on a recording medium, such as a compact disc read only memory (CD-ROM), a digital versatile disc read only memory (DVD-ROM), or a universal serial bus (USB) memory. In addition, the association program 110 may have a form being downloaded from an external device via the network.

What is claimed is:

1. An information processing apparatus comprising:
at least one processor,
wherein the processor
acquires input information, which is information input to the information processing apparatus, and biological information of a user, and
performs association processing of associating information on a physical condition, a symptom, health, feeling, emotion, or a disease name of the user in the input information with the biological information at a point in time corresponding to a point in time when the input information is input.

2. The information processing apparatus according to claim 1,
wherein the input information includes a text input to the information processing apparatus or a selection item selected by using the information processing apparatus.

3. The information processing apparatus according to claim 1,
wherein the input information is information input on an application installed in the information processing apparatus.

4. The information processing apparatus according to claim 1,
wherein the input information includes a search keyword input to a search engine for searching for information on a network.

5. The information processing apparatus according to claim 1,
wherein the input information includes a content transmitted to or sent to a specified or unspecified party.

6. The information processing apparatus according to claim 1,
wherein the input information includes information on a product or service purchased or a product or service to be purchased by information communication via a network.

7. The information processing apparatus according to claim 1,
wherein the processor stores the input information and the biological information associated with each other in a storage medium.

8. The information processing apparatus according to claim 1,
wherein the processor acquires the biological information from a measurement device that measures the biological information by communicating with the measurement device.

9. The information processing apparatus according to claim 1,
wherein the processor
acquires the input information and the biological information for each of a plurality of the users, and
performs the association processing for each user.

10. The information processing apparatus according to claim 1,
wherein the processor
acquires external input information which is information input to an external device by a third party who is permitted to intervene in the association processing, and
further performs association processing of associating information on a physical condition, a symptom, health, feeling, emotion, or a disease name of the user in the external input information with the biological information at a point in time corresponding to a point in time when the external input information is input.

11. The information processing apparatus according to claim 1,
wherein the processor acquires a result of examination performed on the user as the biological information.

12. An information processing method executed by at least one processor provided in an information processing apparatus, the method comprising:
acquiring, by the processor, input information, which is information input to the information processing apparatus, and biological information of a user; and
performing, by the processor, association processing of associating information on a physical condition, a symptom, health, feeling, emotion, or a disease name of the user in the input information with the biological information at a point in time corresponding to a point in time when the input information is input.

13. A non-transitory computer-readable storage medium storing an information processing program causing at least one processor provided in an information processing apparatus to execute:
acquiring input information, which is information input to the information processing apparatus, and biological information of a user; and
performing association processing of associating information on a physical condition, a symptom, health, feeling, emotion, or a disease name of the user in the input information with the biological information at a point in time corresponding to a point in time when the input information is input.
